(19) United States
(12) Patent Application Publication
(10) Pub. No.: US 2021/0251869 A1
(43) Pub. Date: Aug. 19, 2021

(54) SKIN TREATMENT METHODS

(71) Applicant: Amazentis SA, Ecublens (CH)

(72) Inventors: Christopher L. Rinsch, Morges (CH); Penelope Andreux, Eclepens (CH); Anurag Singh, Crissier (CH)

(21) Appl. No.: 17/104,155

(22) Filed: Nov. 25, 2020

Related U.S. Application Data
(63) Continuation of application No. 15/915,777, filed on Mar. 8, 2018.

(30) Foreign Application Priority Data
Mar. 8, 2017 (GB) GB 1703734.2
Mar. 9, 2017 (GB) GB 1703768.0

(51) Int. Cl. A61K 8/49; A61P 17/02; A61P 17/18; A61K 31/366; A61P 17/04; A61K 9/06; A61K 9/00; A61P 17/00; A61P 17/06; A61P 17/12; A61P 17/16; A61Q 17/04; A61Q 19/02

(52) U.S. Cl. CPC A61K 8/498; A61P 17/02; A61P 17/18; A61K 31/366; A61P 17/04; A61K 9/06; A61K 2800/30; A61P 17/00; A61P 17/06; A61P 17/12; A61P 17/16; A61Q 17/04; A61Q 19/02; A61K 9/0014

(57) ABSTRACT

The invention provides a compound of formula (I)

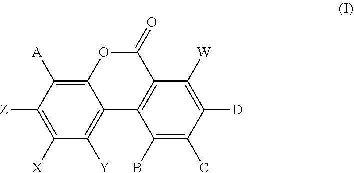

wherein:
A, B, C, D, W, X, Y and Z are each independently selected from H and OH;
or a salt thereof;
for use in the treatment and/or prevention of a skin condition in a subject, wherein the compound of formula (I) or salt thereof is administered topically. The invention further provides topical compostions of a compound of formula (i) and processes for the preparation of said compositions.

### RELATED APPLICATIONS

This application is a continuation of U.S. patent application Ser. No. 15/915,777, filed Mar. 8, 2018; which claims the benefit of priority to GB Patent Application No. 1703768.0, filed Mar. 9, 2017; and GB Patent Application No. 1703734.2, filed Mar. 8, 2017.

### FIELD

The present disclosure relates to methods for treating and preventing skin conditions and disorders such as melasma, which involve topical administration of urolithins. The present disclosure also relates to cosmetic uses, such as skin lightening. Topical compositions comprising the urolithins are also provided, as are processes for making such compositions, e.g. involving the use of micronized urolithins.

### BACKGROUND

Skin conditions associated with hyperpigmentation, such as melasma, are a problem affecting many people. Melasma tends to manifest as brown, tan or grey spots on the face and most commonly affects women in the age range of 20-50 years old, although cases can occur in males also. Factors which can contribute to the likelihood and severity of melasma include exposure to sunshine/UV radiation, pregnancy, and exposure to hormonal drugs including contraceptive agents. Melasma is also referred to as chloasma and mask of pregnancy. Treatments include skin-lightening agents such as hydroquinone or kojic acid, dermabrasion and laser treatment.

However, hydroquinone and kojic acid-containing products can be associated with causing irritation, inflammation and/or contact dermatitis, and in some cases hydroquinone can cause increased skin-darkening. To alleviate skin irritation, steroid active ingredients sometimes need to be used in conjunction with a skin-lightening agent. Chemical peels (e.g. glycolic acid-based) can also be used, but in some instances scarring and/or hypopigmentation may occur. Consequently, there remains a need for effective therapies to treat melasma, as well as other skin conditions associated with hyperpigmentation, for example liver spots/lentigo, and there also remains a need for further agents which can protect against the damaging effects of sunlight and other environmental conditions to which skin is exposed.

Many individuals choose undergo skin lightening or skin bleaching procedures for cosmetic reasons, for example to achieve evenness of skin tone. It would also be desirable to provide improved or alternative approaches for such treatments.

Urolithins are a group of ellagitannin- and ellagic acid-derived metabolites produced by, e.g., mammalian colonic microflora. Urolithins have been proposed as being compounds useful for promoting longevity, see for example WO2014/004902, in the name of Amazentis SA. Compositions for oral administration of urolithins have been proposed, for example WO2014/004092 describes animal experiments in which urolithin A was mixed with food.

### SUMMARY

The present disclosure provides a method of prevention and/or treatment of a skin condition, disease or disorder in a subject, comprising:

topically administering an effective amount of a compound of formula (I)

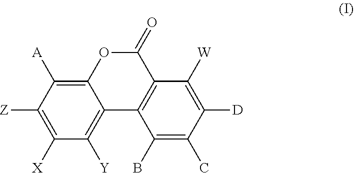

wherein:
A, B, C, D, W, X, Y and Z are each independently selected from H and OH;
or a salt thereof;
to the subject.

The present disclosure also provides a compound of formula (I)

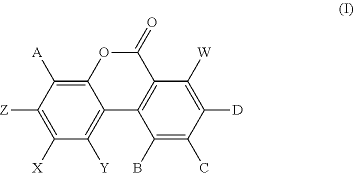

wherein:
A, B, C, D, W, X, Y and Z are each independently selected from H and OH;
or a salt thereof;
for use in the treatment and/or prevention of a skin condition in a subject, wherein the compound of formula (I) or salt thereof is administered topically.

The present disclosure also provides use of a compound of formula (I)

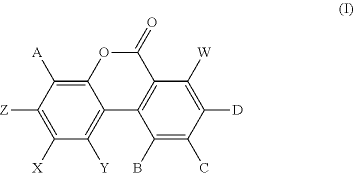

wherein:
A, B, C, D, W, X, Y and Z are each independently selected from H and OH;
or a salt thereof;
for the manufacture of a medicament for the treatment and/or prevention of a skin condition in a subject, wherein the compound of formula (I) or salt thereof is administered topically.

The present disclosure also provides a method of skin bleaching and/or lightening skin colour and/or lightening skin tone of a subject, comprising:

topically administering an effective amount of a compound of formula (I)

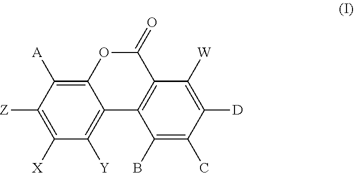

wherein:
A, B, C, D, W, X, Y and Z are each independently selected from H and OH;
or a salt thereof;
to the subject.

A method of skin bleaching and/or lightening skin colour (skin whitening) and/or lightening skin tone of a subject comprises depigmentation.

The present disclosure also provides a composition for topical administration, comprising:

a) a compound of formula (I)

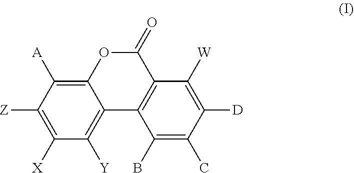

wherein:
A, B, C, D, W, X, Y and Z are each independently selected from H and OH;
or a salt thereof;
and
b) at least one excipient which is suitable for topical administration.

The present disclosure also provides a process for obtaining a composition for topical administration, the composition comprising:

a) a compound of formula (I)

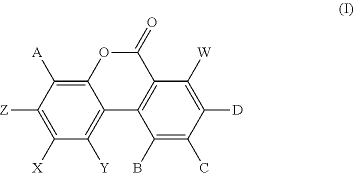

wherein:
A, B, C, D, W, X, Y and Z are each independently selected from H and OH;
or a salt thereof;
and
b) at least one excipient which is suitable for topical administration;
and the process comprising:
providing micronized compound of formula (I) or salt thereof; and
admixing the micronized compound of formula (I) or salt thereof and at least one excipient suitable for topical administration.

The present disclosure also provides a process for obtaining a composition for topical administration, the composition comprising:

a) a compound of formula (I)

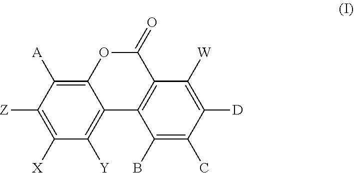

wherein:
A, B, C, D, W, X, Y and Z are each independently selected from H and OH;
or a salt thereof;
and
b) at least one excipient which is suitable for topical administration;
and the process comprising:
providing micronized compound of formula (I) or salt thereof having a D₅₀ in the range of from 0.5 to 50 μm and a D₉₀ in the range of from 5 to 100 μm; and
admixing the micronized compound of formula (I) or salt thereof and at least one excipient suitable for topical administration.

In another embodiment of the invention, there is provided a composition obtainable by a process of the invention.

### SUMMARY OF THE FIGURES

FIG. 1 shows the results of a skin cell viability assay performed in the EpiDerm™ tissues after 96 hours of treatment with water (NC), DMSO 0.5%, Urolithin A (UA) at 25, 50 and 100 μM. The dashed line corresponds to 90% of viability, the threshold below which compounds are considered as irritant.

FIG. 2 is a top view of MelanoDerm™ tissues after 14 days of treatment with water, kojic acid at 2%, DMSO 0.2% and Urolithin A at 50 and 100 μM.

FIG. 3 represents L values measured from MelanoDerm™ tissues after 4, 7, 11 and 14 days of treatment with water, kojic acid 2%, DMSO 0.2% and Urolithin A at 50 and 100 μM. Statistics were computed using a two-way ANOVA test. **P<0.01, ***P<0.001 correspond to a Holm-Sidak's post-hoc test for multiple comparison of each group against the negative control group.

FIG. 4 represents melanin content measured from MelanoDerm™ tissues after 14 days of treatment with water, kojic acid 2%, DMSO 0.2% and Urolithin A at 50 and 100 μM. Statistical significance was performed using a one-way ANOVA. ***P<0.001 corresponding to a Dunnett post-hoc test for multiple comparison of each group against the negative control. The P value of an additional Student t test to compare Kojic acid 2% and Urolithin A 100 μM groups is also reported.

### DETAILED DESCRIPTION

The present disclosure provides methods of treating and/or preventing skin conditions involving topical administration of compounds of formula (I), i.e. urolithins. Whilst urolithins have been dosed orally, in many settings the compounds suffer from limited bioavailability. It has also been found that urolithin compounds are highly water-insoluble. The inventors have found that, despite the above properties, urolithins are suitable for topical administration and demonstrate unexpectedly good results in an in vitro assay for the hyperpigmentation skin condition melasma.

### Compounds of Formula (I) and Salts Thereof

Urolithins are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown above. The structures of some particularly common urolithins are described in Table 1 below, with reference to structure (I).

TABLE 1

| | Substituent of structure (I) | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO2014/004902. Urolithins of any structure according to structure (I) may be used in the methods of the present disclosure.

In one aspect of the uses and methods of the present disclosure, a suitable compound is a compound of formula (I) wherein A, C, D and Z are independently selected from H and OH and B, W, X and Y are all H.

Particularly suitable compounds are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B, C and D are all H, and Z is OH, then the compound is urolithin B. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

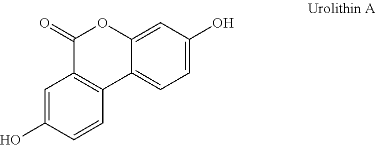 [SMILES: O=c1oc2cc(O)ccc2c2ccc(O)cc12]

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

Those skilled in the art of organic chemistry will appreciate that many organic compounds can form complexes with solvents in which they are reacted or from which they are precipitated or crystallized. These complexes are known as “solvates”. It will be understood by the skilled person that the invention also encompasses solvates of the compounds of formula (I), as well as solvates of salts thereof. Solvates include those where the associated solvent is pharmaceutically acceptable. A hydrate (in which the associated solvent is water) is an example of a solvate.

### Compositions

The methods of the present disclosure involve topical administration of the compound of formula (I) or a salt thereof. Accordingly, the present disclosure also relates to compositions for topical administration, which comprise:

a) a compound of formula (I)

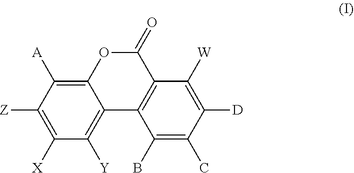

wherein:
A, B, C, D, W, X, Y and Z are each independently selected from H and OH;

or a salt thereof;

and

b) at least one excipient which is suitable for topical administration.

In some preferred embodiments, the compound of formula (I) used in the compositions is urolithin A.

In some preferred embodiments, the compound of formula (I) or salt thereof (e.g. urolithin A) used to produce the compositions of the present disclosure has been micronized. Micronization of the compound results in good activity, and results in a topical composition which is easily applicable to the skin.

If micronized compound of formula (I) or salt thereof is used, then preferably, the compound or salt (e.g. urolithin A) has a D₅₀ size of under 100 μm—that is to say that 50% of the compound or salt by mass has a particle diameter size of under 100 μm. More preferably, the compound or salt (e.g. urolithin A) has a D₅₀ size of under 75 μm, for example under 50 μm, for example under 25 μm, for example under 20 μm, for example under 10 μm. More preferably, the compound or salt (e.g. urolithin A) has a D₅₀ in the range 0.5-50 μm, for example 0.5 to 20 μm, for example 0.5 to 10 μm, for example 1.0 to 10 μm, for example 1.5 to 7.5 μm, for example 2 to 7 μm, for example 2.8 to 5.5 μm. In some embodiments the compound or salt (e.g. urolithin A) has a D₅₀ of about 3.9 μm. In some embodiments the compound or salt (e.g. urolithin A) has a D₅₀ of about 7.1 μm. Preferably, the compound or salt (e.g. urolithin A) has a D₉₀ size of under 100 μm. More preferably, the compound or salt (e.g. urolithin A) has a D₉₀ size of under 75 μm, for example under 50 μm, for example under 25 μm, for example under 20 μm, for example under 15 μm. The compound or salt (e.g. urolithin A) preferably has a D₉₀ in the range 5 to 100 μm, for example 5 to 50 μm, for example 5 to 20 μm, for example 7.5 to 15 μm, for example 8 to 20 μm, for example 8.2 to 16.0 μm. In some embodiments the compound or salt (e.g. urolithin A) has a D₉₀ of about 11.5 μm. In some embodiments the compound or salt (e.g. urolithin A) has a D₉₀ of about 13.5 μm. Preferably, the compound or salt (e.g. urolithin A) has a D₁₀ in the range 0.5 to 2 μm, or in the range 0.5-1.0 μm. Preferably, the compound or salt (e.g. urolithin A) has a D₁₀ size of under 50 μm. More preferably, the compound or salt (e.g. urolithin A) has a D₁₀ size of under 25 μm, for example under 20 μm, for example under 15 μm, for example under 10 μm, for example under 5 μm, for example under 2 μm. The compound or salt (e.g. urolithin A) preferably has a D₁₀ in the range 0.1 to 25 μm, for example 0.1 to 10 μm, for example 0.5 to 5 μm, for example 0.5 to 2 μm, for example 0.5 to 1.0 μm. In some embodiments the compound or salt (e.g. urolithin A) has a D₁₀ of about 1.2 μm. In some embodiments the compound or salt (e.g. urolithin A) has a D₁₀ of about 0.7 μm. In some embodiments, the compound of formula (I) or salt thereof (e.g. urolithin A) has a D₅₀ in the range of from 0.5 to 50 μm, and a D₉₀ in the range of from 5 to 100 μm. In some embodiments, the compound of formula (I) or salt thereof (e.g. urolithin A) has a D₉₀ in the range of from 8 to 20 μm, a D₅₀ in the range of from 2 to 7 μm and a D₁₀ in the range of from 0.5 to 2 μm. In some embodiments, the compound of formula (I) or salt thereof (e.g. urolithin A) has a D₉₀ in the range 8.2 to 16.0 μm, a D₅₀ in the range 2.8 to 5.5 μm and a D₁₀ in the range 0.5 to 1.0 μm. Micronisation can be achieved by methods established in the art, for example compressive force milling, hamermilling, universal or pin milling, or jet milling (for example spiral jet milling or fluidised-bed jet milling) may be used. Jet milling is especially suitable. Methods for determining particle size are known in the art, for example equipment such as a Beckman Counter L3 13 320 or Malvern Mastersizer 2000 may be used. In some embodiments, particle size (e.g. D₁₀, D₅₀, and/or D₉₀ values) may be determined using a Malvern Mastersizer 2000.

In an alternative embodiment, the compound or salt (e.g. urolithin A) has a D₅₀ size of not more than 75 μm, for example not more than 50 μm, for example not more than 25 μm, for example not more than 20 μm, for example not more than 10 μm.

In an alternative embodiment, the compound or salt (e.g. urolithin A) has a D₉₀ size of not more than 75 μm, for example not more than 50 μm, for example not more than 30 μm for example not more than 25 μm, for example not more than 20 μm, for example not more than 15 μm.

In an alternative embodiment, the compound or salt (e.g. urolithin A) has a D₁₀ size of not more than 25 μm, for example not more than 20 μm, for example not more than 15 μm, for example not more than 10 μm, for example not more than 5 μm, for example not more than 2 μm.

Accordingly, in some embodiments the composition is obtainable by a process comprising
providing micronized compound of formula (I) or salt thereof (such as urolithin A), e.g. having a D₅₀ in the range of from 0.5 to 50 μm, and a D₉₀ in the range of from 5 to 100 μm; and
admixing the micronized compound of formula (I) or salt thereof and at least one excipient suitable for topical administration.

Accordingly, there is also provided a process for obtaining a composition for topical administration, the composition comprising:

a) a compound of formula (I)

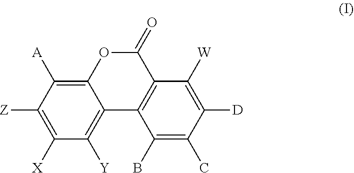

wherein:
A, B, C, D, W, X, Y and Z are each independently selected from H and OH; or a salt thereof;
and
b) at least one excipient which is suitable for topical administration;
and the process comprising:
micronizing a compound of formula (I) or a salt thereof; and
admixing the micronized compound of formula (I) or salt thereof and at least one excipient suitable for topical administration. In some preferred embodiments, the compound is urolithin A.

Accordingly, there is also provided a process for obtaining a composition for topical administration, the composition comprising:

a) a compound of formula (I)

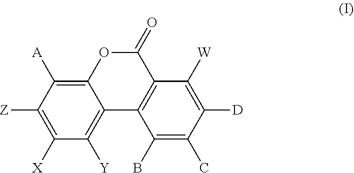

wherein:
A, B, C, D, W, X, Y and Z are each independently selected from H and OH;
or a salt thereof;
and
b) at least one excipient which is suitable for topical administration;
and the process comprising:
micronizing a compound of formula (I) or a salt thereof, thereby producing micronized compound of formula (I) or salt thereof having a D₅₀ in the range of from 0.5 to 50 μm, and a D₉₀ in the range of from 5 to 100 μm; and
admixing the micronized compound of formula (I) or salt thereof and at least one excipient suitable for topical administration. In some preferred embodiments, the compound is urolithin A.

As discussed above, compounds of formula (I) are metabolites produced by the action of mammalian gut microbiota on ellagitannins and ellagic acid, which are polyphenol compounds commonly found in foods such as pomegranates, nuts and berries. Typically the compound of formula (I) or salt thereof used in the compositions, methods and uses of the present disclosure is in purified form, most commonly obtained via chemical synthesis and purification. Thus, in some embodiments, the compound of formula (I) or salt thereof used to produce the compositions are at least 90% pure by weight, at least 95% pure by weight, at least 97% pure by weight, at least 98% pure by weight, at least 99% pure by weight, or at least 99.5%. In some embodiments, less than 10% by weight, less than 5% by weight, less than 3% by weight, less than 2% by weight, or less than 1% by weight of the polyphenols present in the composition are other than compound of formula (I) or salt thereof. In some embodiments the composition is substantially free from polyphenols other than the compound of formula (I) or salt thereof.

The compositions of the present disclosure are for topical application, and contain excipients suitable for topical application, e.g. which facilitate delivery of the active compound to the site of action, which are compatible with the active compound and provide for good chemical and physical stability, and which are safe and have no or low irritancy. Most commonly the composition is a semi-solid or liquid composition. In some embodiments, the composition is a liquid. In other embodiments the composition is a semi-solid. In some embodiments, the composition is in the form of a solution, a suspension, an emulsion, a gel, a solid or a liposome formulation.

Typically the composition (for example a cream) will contain a topically acceptable carrier or vehicle which may for example, make up from 50% to 99.9995%, or from 60% to 99.9995%, or from 70% to 99.9995%, or from 80% to 99.9995%, or from 90% to 99.9995%, or from 95% to 99.9995%, from 50% to 99.995%, or from 60% to 99.995%, or from 70% to 99.995%, or from 80% to 99.995%, or from 90% to 99.995%, or from 95% to 99.995%, or from 70% to 99.99%, or from 80% to 99.99%, or from 90% to 99.99%, or from 95% to 99.99%, or from 70% to 99.9%, or from 80% to 99.9%, or from 90% to 99.9%, or from 95% to 99.9%, or from 98% to 99.9%, or from 80% to 99%, or from 90% to 99%, or from 95% to 99%, or from 98% to 99% by weight of the composition. In some embodiments the compound of formula (I) or salt thereof is present in the composition in an amount in the range of from 0.0005% to 50%, from 0.0005% to 40%, from 0.0005% to 30%, from 0.0005% to 20%, from 0.0005% to 10%, from 0.0005% to 1%, from 0.005% to 50%, from 0.005% to 40%, from 0.005% to 30%, from 0.005% to 20%, from 0.005% to 10%, from 0.005% to 1%, from 0.01% to 30%, from 0.01% to 20%, from 0.01% to 10%, from 0.01% to 5%, from 0.1% to 30%, from 0.1% to 20%, from 0.1% to 10%, from 0.1% to 5%, from 0.1% to 2%, from 1% to 20%, from 1% to 10%, from 1% to 5%, or from 1% to 2% by weight. In some embodiments the compound of formula (I) or salt thereof is present in the composition at a concentration in the range of from 1 μM to 2.5M, from 1 μM to 1M, from 1 μM to 100 mM, from 1 μM to 10 mM, from 1 μM to 100 μM, from 10 μM to 2.5M, from 10 μM to 1M, from 10 μM to 100 mM, from 10 μM to 10 mM, from 10 μM to 5 mM, from 10 μM to 1 mM, from 10 μM to 500 μM, from 10 μM to 250 μM, from 25 μM to 2.5M, from 25 μM to 1M, from 25 μM to 100 mM, from 25 μM to 10 mM, from 25 μM to 5 mM, from 25 μM to 1 mM, from 25 μM to 500 μM, from 25 μM to 250 μM, from 100 μM to 2.5M, from 100 μM to 1M, from 100 μM to 100 mM, from 100 μM to 10 mM, from 100 μM to 1 mM, from 500 μM to 2.5M, from 500 μM to 1M, from 500 μM to 100 mM, from 500 μM to 10 mM, or from 500 μM to 1 mM. Compositions containing different concentrations of compound of formula (I) or salt thereof may for example be used. For example, a composition for cosmetic application, or an over-the-counter compositions intended for patients having less severe symptoms may contain a lower concentration of active agent, and a prescription-only composition, e.g. intended for treatment of severe symptoms, may contain a higher concentration of active agent.

In a further embodiments the compound of formula (I) or salt thereof is present in the composition at a concentration in the range of from 0.01 μM to 100 mM, from 0.01 μM to 10 mM, from 0.01 μM to 1 mM, from 0.01 μM to 100 μM, from 0.1 μM to 500 μM, from 0.1 μM to 100 μM, or from 1 μM to 50 μM.

In one embodiment, topical compositions of the inventions (such as creams) comprise 0.0001% to 5%; of a compound of formula (I) or salt thereof, for example, 00001% to 1%, such as 0.0001% to 0.1%, such as 0.0001% to 0.01%. In a further embodiment, topical compositions of the inventions (such as creams) comprise 0.001% to 0.5%; of a compound of formula (I) or salt thereof, for example: 0.001% to 0.1%, such as 0.01% to 0.1%; In a further embodiment topical compositions of the invention (such as creams) comprise 0.005% to 0.05%; of a compound of formula (I) or salt thereof, for example, 0.001% to 0.01%. Examples of constituents of topical compositions include oils, glycerides (including tri-, di- and/or mono-glycerides), organic solvents (e.g. alcohols), water, waxes, greases, surfactants, emollients, moisturising agents, skin conditioning agents, thickeners, emulsifiers, gelling agents, foaming agents, preservatives, buffering agents, chelating agents, opacifiers, flavouring agents, coloring agents, fragrances or perfumes, additional therapeutically active agents, and additional cosmetically active agents. Mixtures of the above may be used.

In some embodiments, the composition comprises an oil and/or lipid component. Examples of oils that can be used in a formulation for topical application are well known in the art and they include cottonseed, groundnut, corn, germ, olive, castor, soybean, mineral, sesame and evening primrose oils. In some embodiments the composition comprises an oil in an amount of up to 90%, up to 80%, up to 70%, up to 60%, up to 50%, up to 40%, up to 30%, up to 20%, up to 10%, up to 5%, from 1% to 50%, from 1% to 40%, from 1% to 30%, from 1% to 20%, from 1% to 10%, from 5% to 50%, from 5% to 40%, from 5% to 30%, from 5% to 20%, from 5% to 10%, from 10% to 50%, from 10% to 40%, from 10% to 30%, from 10% to 30% by weight of the composition.

In some embodiments, the composition comprises an organic solvent. Examples of organic solvents include alcohol solvents (e.g. ethanol, isopropanol, ethylene glycol, propylene glycol), pyrrolidones (e.g. N-methylpyrrolidinone) and DMSO. In some embodiments the composition comprises an organic solvent in an amount of up to 90%, up to 80%, up to 70%, up to 60%, up to 50%, up to 40%, up to 30%, up to 20%, up to 10%, up to 5%, up to 3%, up to 2%, up to 1%, from 0.1% to 20%, from 0.1% to 15%, from 0.1% to 10%, from 0.1% to 5%, from 0.1% to 3%, from 0.1% to 2%, from 0.1% to 1%, from 1% to 50%, from 1% to 40%, from 1% to 30%, from 1% to 20%, from 1% to 10%, from 5% to 50%, from 5% to 40%, from 5% to 30%, from 5% to 20%, from 5% to 10%, from 10% to 50%, from 10% to 40%, from 10% to 30%, from 10% to 20% by weight of the composition.

In some embodiments, the composition comprises a medium chain triglyceride. Medium chain triglycerides are compounds of formula CH₂(OR¹)—CH(OR²)—CH₂(OR³) where R¹, R² and R³ are medium chain fatty acid groups, generally of formula —C(═O)(CH₂)ₙCH₃ where n is in the range 4 to 10, for example 6 to 8. Medium-chain fatty acids are fatty acids which have an aliphatic tail of 6-12 carbon atoms. The aliphatic tail is predominantly saturated. Particular medium-chain fatty acids include caproic acid (hexanoic acid, C6:0), caprylic acid (octanoic acid, C8:0), capric acid (decanoic acid, 010:0) and lauric acid (dodecanoic acid, C12:0). Myristic acid (tetradecanoic acid, C14:0) can also be present in minor amounts. Medium-chain triglycerides most commonly used generally have a mixture of triglycerides of caprylic acid and capric acid, and contain 95% or greater of saturated fatty acids. The medium chain triglyceride component in the composition of the invention can consist of a homogeneous, single medium chain triglyeride compound type; more commonly, the medium chain triglyceride component in the composition of the invention is a mixture of two or more different medium chain triglyeride compounds.

The European Pharmacopoeia describes medium-chain triglycerides as the fixed oil extracted from the hard, dried fraction of the endosperm of Cocos nucifera L. (coconut) or from the dried endosperm of Elaeis guineenis Jacq. (African oil palm). The European Pharmacopoeia and the USPNF both have specifications for medium-chain triglycerides that require the presence of particular fatty acids is as follows: caproic acid (C6)≤2.0% caprylic acid (C8) 50.0-80.0%; capric acid (C10) 20.0-50.0%; lauric acid (C12)≤3.0%, and myristic acid (C14)≤1%.

In particular, medium-chain triglycerides for use in compositions of the invention comprise a mixture of triglycerides with fatty acid chains present in the following proportions: C6≤5%; C8 50-70%; C10 30-50%; and C12≤12%, for example C6≤0.5%, C8 55-65%; C10 35-45%; and C12≤1.5%.

Medium-chain triglycerides for use in compositions of the present invention may be obtained from any suitable source. In some embodiments the composition comprises a medium-chain triglyceride in an amount of up to 90%, up to 80%, up to 70%, up to 60%, up to 50%, up to 40%, up to 30%, up to 20%, up to 10%, up to 5%, from 1% to 50%, from 1% to 40%, from 1% to 30%, from 1% to 20%, from 1% to 10%, from 5% to 50%, from 5% to 40%, from 5% to 30%, from 5% to 20%, from 5% to 10%, from 10% to 50%, from 10% to 40%, from 10% to 30%, from 10% to 30% by weight of the composition.

In some embodiments, the composition comprises an emollient, which is a material used for prevention and/or relief of dryness. Examples of emollients include vegetable oils, mineral oils, silicone oils, fatty acid esters, and alcohols such as 1-hexadecanol.

In some embodiments, the composition comprises an emulsifier. Examples of emulsifiers include PPG-1-PEG-9 Lauryl Glycol Ether (Trade name: Eumulgin L), PEG-60 Hydrogenated Castor Oil (Trade name: Cremophor CO 60), Cetyl Alcohol and Glyceryl Stearate and PEG-75 Stearate and Ceteth-20 and Steareth-20 (Trade name: Emulium Delta), Cetearyl Alcohol (Trade name: Nafoi 1618), Hydroxyethyl Acrylate/Sodium Acryloyldimethyl Ta urate Copolymer and water and Squalane and Polysorbate 60 and Sorbitan Isostearate (Trade name: Simulgel NS).

In some embodiments, the composition comprises a thickener. Examples of thickeners include cross-linked acrylates (e.g. Carbopol 982), hydrophobically-modified acrylates (e.g. Carbopol 1382), cellulosic derivatives (such as sodium carboxymethylcellulose, hydroxypropylmethylcellulose, hydroxyethylcellulose, ethyl cellulose, hydroxymethylcellulose) and natural gums (e.g. guar, xanthan, sclerotium, carrageenan, pectin). When used, typically a thickener is used in an amount of up to 5%, or up to 1%, by weight of the composition.

An example of a metal chelator or sequestrant is a salt of EDTA (ethylenediamine tetraacetic acid).

Where a surfactant is used, it is typically used in an amount of up to 40%, or up to 30%, or up to 20%, or up to 10%, by weight of the composition.

In some embodiments, the composition contains a further active agent in addition to the compound of formula (I) or salt thereof. For example, it may contain an additional active agent useful for treating or preventing a skin condition such as melisma, e.g. such as hydroquinone or kojic acid.

Examples of suitable types of composition include creams, pastes, ointments, solutions, lotions, foams, mousses, gels, sticks and sprays. Further examples of suitable compositions include creams, dispersions, emulsions, gels, ointments, lotions, milk, mousses, sprays, or tonics.

In some embodiments, the composition is in the form of a cream or lotion, e.g. a skin cream. Creams typically take the form of an oil and water emulsion, classified as oil in water (o/w) or water in oil (w/o) emulsions. In some embodiments the composition is a cream which is an oil in water emulsion. In some embodiments the composition is a cream comprising from 5% to 50% of an oil (e.g. an emollient), and from 45% to 85% of water. Topical creams typically additionally contain emulsifiers and/or thickeners.

A lotion typically refers to a liquid preparation containing the active ingredient suspended or dissolved in the liquid carrier. Lotions may for example be aqueous- and/or organic solvent- (e.g. alcohol-) based formulations.

In some embodiments, the composition is in the form of an ointment. Ointments are typically semi-solid preparations of hydrocarbons (such as petrolatum, mineral oil, paraffins, synthetic hydrocarbons), and which are often viscous and/or greasy. In many cases, ointments may contain little or no water. In some embodiments the ointment comprises a hydrocarbon/oil base, an emollient (e.g. about 2% to 10% by weight), and a thickening agent (e.g. about 1% to 2% by weight). In some embodiments, the composition is in the form of a gel. Gels typically contain a gelling agent (such as a natural gum, an acrylate polymer/copolymer or a cellulose derivative) and a suitable liquid component (e.g. an organic solvent such as an alcohol. In some embodiments, the composition is in the form of a paste. Pastes are typically a mixture of a powder and a liquid or semi-solid carrier, such as an ointment.

The present disclosure relates for example to protection of skin from damage caused by the environment, e.g. to the use of the compound of formula (I) as a nutrient to protect against damage caused by sunlight/UV radiation. Accordingly, in some embodiments, the topical composition comprising the compound of formula (I) is a sunscreen composition, e.g. a cream, lotion or spray. Such compositions typically contain, in addition to the compound of formula (I) or salt thereof, a physical and/or chemical sunscreen, such as a UV-blocking agent. In some embodiments, the sunscreen composition comprises a physical sunscreen, e.g. such as titanium dioxide or zinc oxide. In some embodiments, the sunscreen composition comprises a chemical sunscreen, e.g. such as oxybenzone, avobenzone, octisalate, octocrylene, homosalate and/or octinoxate.

The UV-blocking agent can be an organic compound that absorbs light in the UV region at one or more wavelengths from 290 nanometers (nm) to 400 nm. For example, the UV-blocking agent can exhibit a molar extinction coefficient of at least 10,000 mol⁻¹ L cm⁻¹ (e.g., at least 25,000 mol⁻¹ L cm⁻¹, at least 50,000 mol⁻¹ L cm⁻¹, at least 75,000 mol⁻¹ L cm⁻¹, or at least 100,000 mol⁻¹ L cm⁻¹) for at least one wavelength within the range of from 290 nm to 400 nm.

In some embodiments, the UV-blocking agent can be an organic compound that absorbs light in the UV-B region at one or more wavelengths from 290 nm to 320 nm (i.e., a UV-B blocking agent). For example, the UV-blocking agent can exhibit a molar extinction coefficient of at least 10,000 mol⁻¹ L cm⁻¹ (e.g., at least 25,000 mol⁻¹ L cm′1, at least 50,000 mol⁻¹ L cm⁻¹, at least 75,000 mol⁻¹ L cm⁻¹, or at least 100,000 mol⁻¹ L cm⁻¹) for at least one wavelength within the range of from 290 nm to 320 nm.

In some cases, the UV-blocking agent can exhibit a molar extinction coefficient of at least 10,000 mol⁻¹ L cm⁻¹ at all wavelengths within the range of from 290 nm to 320 nm.

In some embodiments, the UV-blocking agent can be an organic compound that absorbs light in the UV-A region at one or more wavelengths from 320 nm to 400 nm (i.e., a UV-A blocking agent). For example, the UV-blocking agent can exhibit a molar extinction coefficient of at least 10,000 mol⁻¹ L cm⁻¹ (e.g., at least 25,000 mol⁻¹ L cm⁻¹, at least 50,000 mol⁻¹ L cm⁻¹, at least 75,000 mol⁻¹ L cm⁻¹, or at least 100,000 mol⁻¹ L cm⁻¹) for at least one wavelength within the range of from 320 nm to 400 nm.

In some cases, the UV-blocking agent can exhibit a molar extinction coefficient of at least 10,000 mol′1 L cm′1 at all wavelengths within the range of from 320 nm to 400 nm.

Examples of suitable UV-blocking agents include, for example, p-aminobenzoic acid, padiate O, phenylbenzimidazole sulfonic acid, cinoxate, dixoybenzone, oxybenzone, homosalate, menthyl anthranilate, octocrylene, octyl methoxycinnamate, octyl salicylate, sulisobenzone, trolamine salicylate, avobenzone, ecamsule, 4-methylbenzylidene camphor, bisoctrizole, bemotrizinol, bisdisulizole disodium, tris-biphenyl triazine, drometrizole, trisiloxane, benzophenone-9, ethylhexyl triazone, diethylamino hydroxybenzoyl hexyl benzoate, iscotrizinol, polysilicone-15, amiloxate, and combinations thereof. In some embodiments, the UV-blocking agent can be p-aminobenzoic acid, padiate 0, phenylbenzimidazole sulfonic acid, cinoxate, dixoybenzone, oxybenzone, homosalate, menthyl anthranilate, octocrylene, octyl 15 methoxycinnamate, octyl salicylate, sulisobenzone, trolamine salicylate, avobenzone, ecamsule, or a combination thereof. In certain embodiments, the UV-blocking agent can be avobenzone, oxybenzone, or a combination thereof.

The sunscreen agent can be present in the composition in an amount of from 0.5% to 10% by weight, based on the total weight of the composition. The composition can be formulated to exhibit an SPF of at least 15 (e.g., at least 30), as measured using the international standard ISO 24444: 2010(E).

The present disclosure also relates for example to cosmetic skin bleaching and/or skin lightening. Accordingly, in some embodiments, the topical composition comprising the compound of formula (I) is a make-up composition, e.g. a foundation composition.

It has surprisingly been found that urolithin A is especially soluble in a topical cream composition including lipophilic excipients, e.g. an oil and/or a medium chain triglyceride, Such a composition is homogeneous in its appearance with no visible solid urolithin A after admixing, and has a smooth feel on the skin When micronized to urolithin A was mixed with skin cream, it formed a smooth mixture with darkened colour, indicating that the urolithin had either dissolved in a component of the cream or become suspended in the cream matrix.

Accordingly, in some embodiments, the topical composition comprises an organic solvent which is suitable for topical administration. In some embodiments, the composition comprises an oil and/or lipid component.

In some embodiments, the topical composition comprises a mono-, di- and/or tri-glyceride. In some embodiments, the composition comprises a medium chain triglyceride.

In some embodiments, the water content of the composition is low, for example the composition may contain less than 20%, less than 10%, less than 5%, less than 2% or less than 1% water by weight. In some embodiments, the composition is substantially free from water. However, in some other embodiments, the composition may contain a significant proportion of water, for example where the composition contains a mixture of water and oil or water and organic solvent, e.g. together with an emulsifier.

The present disclosure concerns compositions for topical administration. Compounds of formula (I) have previously been administered orally. It will be appreciated that some excipients which are suitable for topical formulation of actives are unsuitable for oral administration, and the present disclosure includes formulations which include one or more excipients which are unsuitable for oral administration.

In some embodiments, the composition comprises a skin penetration enhancer, to aid delivery of the active ingredient into and/or through the skin. Examples of skin penetration enhancers include sulfoxides (such as DMSO), pyrrolidones, terpenes, fatty acids, alcohols, glycols, glycol ethers and glycerides.

In some embodiments the composition (e.g. a cream) comprises the compound of formula (I) or salt thereof at a level of up to 100 mg in a 1 ml portion of composition (e.g. of topical cream composition comprising a lipophilic component such as an oil and/or medium chain triglyceride), so up to 100 mg per ml. Thus the composition may contain, for example compound of formula (I) or a salt thereof (e.g. urolithin A) in an amount in the range of from 0.001 to 100 mg/ml, from 0.01 to 100 mg/ml, from 0.05 to 100 mg/ml, from 0.1 to 100 mg/ml, from 5 to 100 mg/ml, from 10 to 100 mg/ml, from 0.01 to 50 mg/ml, from 0.05 to 50 mg/mL, from 0.1 to 50 mg/ml, from 0.5 to 50 mg/ml, from 1 to 50 mg/ml, from 5 to 50 mg/ml, from 0.001 to 0.1 mg/ml, from 0.01 to 10 mg/ml, from 0.05 to 10 mg/ml, from 0.1 to 10 mg/ml, from 0.5 to 10 mg/ml, from 1 to 10 mg/ml, from 0.01 to 5 mg/ml, from 0.05 to 5 mg/ml, from 0.1 to 5 mg/ml, from 0.5 to 5 mg/ml, from 1 to 5 mg/ml, from 0.01 to 1 mg/ml, from 0.05 to 1 mg/ml, from 0.1 to 1 mg/ml, from 0.5 to 1 mg/ml, from 0.01 to 0.5 mg/ml, or from 0.05 to 0.5 mg/ml of composition.

Example topical compositions of the invention include:

| | 1 | 2 | 3 | 4 |
|---|---|---|---|---|
| Compound of | 0.0001% | 0.001% | 0.01% | 0.1% |
| Formula (I) | to 0.001% | to 0.01% | to 0.1% | to 1% |
| (Quality in | | | | |
| finished topical | | | | |
| cream product | | | | |
| expressed as a | | | | |
| weight percent) | | | | |

| | |
|---|---|
| Other | Avene thermal spring water, glycerin, mineral oil |
| components | cetearyl alcohol, evening primrose oil caprylic/capric |
| | triglyceride, cetearyl glucoside, aquaphilus dolomiae |
| | extract, arginine, carbomer, evening primrose oil/palm oil |
| | aminopropanediol esters, glycine, sodium hydroxide, |
| | tocopherol and water (aqua). |

### Uses

Compounds of formula (I) have been proposed as treatments for a variety of conditions associated with inadequate mitochondrial function. The mitochondrion is a central organelle that can drive both cellular life, i.e. by producing energy in the respiratory chain, and death, i.e. by initiating apoptosis. More recently, it was demonstrated that dysfunctional mitochondria can be specifically targeted for elimination by autophagy, a process that has been termed mitophagy. Increasing mitophagy (the removal of dysfunctional mitochondria) is understood to lead to rejuvenation of mitochondria, and improvement in mitochondrial function. It has been found that urolithin A induces mitophagy and increases lifespan in rodents, see Ryu et al, Nature Medicine, 2016, 22, p 879-888.

Unlike previous approaches, which focused on oral administration, it has now been found that, when contacted with skin samples, in an in vitro assay for the hyperpigmentation skin condition melasma, urolithin A displayed unexpectedly good results. At the higher concentration tested, e.g. 100 μM, the urolithins produced faster skin-lightening effects, and resulted in a greater decrease in melanin content, than the use of 2% kojic acid, an agent currently used for therapeutic and cosmetic skin-lightening.

Accordingly the present disclosure provides a method of prevention and/or treatment of a skin condition, disease or disorder in a subject, comprising topically administering an effective amount of the compound of formula (I), or a salt thereof (e.g. urolithin A), to the subject. The present disclosure also includes methods of preventing and/or treating a skin condition, disease or disorder in a subject, comprising topically administering a composition comprising a) a compound of formula (I), or a salt thereof (e.g. urolithin A); and b) at least one excipient which is suitable for topical administration; to the subject.

In some embodiments, the methods result in faster effects on skin pigmentation than administration with current therapies, such as kojic acid.

In some embodiments, the skin condition, disease or disorder, is a skin condition, disease or disorder associated with hyperpigmentation. In some embodiments, the skin condition, disease or disorder is a skin condition, disease or disorder associated with inadequate mitochondrial activity.

In some embodiments, the skin disease, disorder or condition is selected from the group consisting of melasma, chloasma, mask of pregnancy, hyperpigmentation, skin-aging, liver spots, lentigo, inflammation of the skin, skin irritation, skin infection, warts, psoriasis, and protection of skin from damage caused by the environment and/or therapy. The skin disease, disorder or condition is also selected from melanosis, dermatitis, linea nigra and endocrine diseases such as Addison's and Cushing's syndrome.

In some embodiments, the skin disease, disorder or condition is melasma. Melasma is also referred to as chloasma and mask of pregnancy. Melasma is a common skin condition in adults, especially in women in the age range of 20-50, in which brown, tan or grey pigmentation develops, mainly in the face. Melasma often becomes more noticeable in summer months, and is less noticeable during winter. A number of factors may contribute to the likelihood of having and the severity of melasma, including exposure to sunlight and/or UV light, stress, pregnancy, hypothyroidism, and administration of certain active ingredients, particularly hormonal active ingredients such as oral contraceptive pills. Other factors which can contribute include certain cosmetics. Accordingly, in some embodiments the skin condition, disease or disorder is melasma is selected from the group consisting of stress-related melasma, pregnancy-related melasma, hypothyroidism-associated melasma, melasma associated with administration of an active ingredient, melasma associated with exposure to sunlight and/or UV light, and melasma associated with exposure to a chemical agent.

Test for determining identifying skin-lightening and skin-darkening agents are known in the art, see for example US2008/0249029 and US2012/0128613. For example, as described in US2012/0128613, one epidermal equivalent system useful in performing these types of studies is the MelanoDerm™ system, available commercially from MatTek (Ashland, Mass.). This system contains human normal melanocytes, together with normal, human-derived epidermal keratinocytes, which have been cultured to form a multi layered, highly differentiated model of the human epidermis.

The compounds and compositions of the present disclosure also find use in treating and/or preventing other conditions, diseases or disorders associated with hyperpigmentation, for example age-related hyperpigmentation of the skin, or post-inflammatory hyperpigmentation. Thus in some embodiments, the skin disease, disorder or condition is skin aging, liver spots or lentigo. In some embodiments, the method is for protecting skin from damage caused by the environment, e.g. from damage caused by sunlight/UV rays. In some embodiments, the method is for protecting skin from damage caused by radiation, e.g. UV, beta or gamma radiation including during medical treatment for a condition such as a cancer.

The compounds of the present disclosure also find use in treating and/or preventing skin conditions in which enhancing mitophagy and/or autophagy leads to beneficial effects. In some embodiments, the method is for treating and/or preventing a disease, disorder or condition selected from the group consisting of inflammation of the skin, skin irritation, skin infection, warts and psoriasis.

In some embodiments the methods of the present disclosure are for treatment and/or prevention of medical conditions, i.e. where the subject is an individual that has a disease state or a medical condition or disorder. As referred to herein, a subject that that has a skin disease, condition or disorder, is a subject who has symptoms who has either been diagnosed by a medical practitioner as having a skin disease, disorder or condition, or who, if presented to a medical practitioner, would be diagnosed as having a skin disease, disorder, or condition.

However, in other embodiments, it is envisaged that the compound of formula (I) or salt thereof will be administered to subjects who are not suffering from a particular disease or disorder. For example, the subject may be a healthy individual, i.e. an individual that does not have a skin disorder, disease or condition, who wishes to topically administer the compound of formula (I) or salt thereof to bleach their skin, or lighten their skin colour and/or tone, e.g. for cosmetic reasons, such as providing a smoother and/or more even skin tone or colour. Accordingly, in some embodiments the subject is healthy, and/or the methods of topically administering the compound of formula (I) or salt thereof are cosmetic methods. As referred to herein, a healthy subject is a subject that does not have symptoms which, if presented to a medical practitioner, would be diagnosed as having a skin disease, disorder or condition.

The effective amount of the compound of formula (I) or salt thereof, or of the composition containing the compound, to be taken will vary depending upon the manner of administration, the age, body weight, and general health of the subject. Factors such as the disease state, age, and weight of the subject may be important, and dosage regimens may be adjusted to provide the optimum response.

A subject is any organism which would benefit from topical administration of a compound of formula (I) according to the invention. In some embodiments the subject is a mammal, for example a non-human mammal, for example, cats, dogs, goats, horses and cows, but more preferably the subject is a human. In some embodiments the subject is male. In some embodiments the subject is female. Whilst in certain embodiments the subject may be a child, in other more preferred embodiments the subject is an adult. In some embodiments, the subject may be at least 20, at least 25, at least 30, at least 35, at least 40, at least 45, at least 50, at least 55, at least 60, at least 65, at least 70 or at least 75 years of age. In other embodiments, the subject may be for example in the range of from 18 to 50, from 18 to 40, or from 18 to 30 years of age.

Typically a composition containing the compound will be applied to the affected area or areas of the skin, e.g. it may be spread over the surface and/or rubbed in. Treatment is preferably by way of a series of administrations. For example, topical administration of the compound may be carried out once, twice, or three times daily over a period of time or as often as required. It will also be appreciated that the effective dosage of the compound may increase or decrease over the course of a particular treatment.

As discussed above, administration of urolithin A at 100 μM concentration resulted in faster skin-lightening effects as determined by optical spectrophotometry, and resulted in a greater decrease in melanin content, than 2% kojic acid. Accordingly, in some embodiments, the methods involve less frequent dosing than with current therapies such as kojic acid. For example, in some embodiments, application may only be required once every 2, 3 or 4 days, or for example once per week.

Where, for example, daily administration of the compound of formula (I) or salt thereof (e.g. urolithin A), to a subject is carried out, the amount may for example be in the range of from 0.1 mg to 5 g per day, for example 1 mg to 5 g per day, for example 10 mg to 5 g per day, for example 20 mg to 2500 mg per day, for example 50 mg to 1500 mg per day, for example 100 mg to 1,500 mg per day, for example 150 mg to 1,500 mg per day, for example 200 mg to 1,500 mg per day, for example 250 mg to 1500 mg per day, for example 50 mg to 1000 mg per day, for example 250 mg to 1000 mg per day, for example 10 mg to 1000 mg per day, for example 10 mg to 750 mg per day, for example 20 mg to 500 mg per day, for example 50 mg to 500 mg per day, for example 50 mg to 250 mg per day. In some embodiments the dose is 250 mg/day, in another embodiment the dose is 500 mg/day, in a further embodiment the dose is 750 mg/day, in a further embodiment the dose is 1000 mg/day. In some embodiments the dosage of compound of formula (I) or salt thereof (e.g. urolithin A), may for example be in the range of from 0.01 to 100 mg/kg/day. For example, the dosage of urolithin may be in the range of from 0.1 to 100, 0.2 to 100, 0.2 to 50, 0.2 to 40, 0.2 to 25, 0.2 to 10, 0.2 to 7.5, 0.2 to 5, 0.25 to 100, 0.25 to 25, 0.25 to 25, 0.25 to 10, 0.25 to 7.5, 0.25 to 5, 0.5 to 50, 0.5 to 40, 0.5 to 30, 0.5 to 25, 0.5 to 20, 0.5 to 15, 0.5 to 10, 0.5 to 7.5, 0.5 to 5, 0.75 to 50, 0.75 to 25, 0.75 to 20, 0.75 to 15, 0.75 to 10, 0.75 to 7.5, 0.75 to 5, 1.0 to 50, 1 to 40, 1 to 25, 1 to 20, 1 to 15, 1 to 10, 1 to 7.5, 1 to 5, 2 to 50, 2 to 25, 2 to 20, 2 to 15, 2 to 10, 2 to 7.5, or 2 to 5 mg/kg/day.

The composition containing the compound of formula (I) or salt thereof is typically administered to the affected area or areas of skin. Thus, in some embodiments, on each application (e.g. daily) the compound is topically administered in an amount in the range of from 0.001 to 100 mg/cm² of the skin treated, e.g. from 0.005 to 100, from 0.01 to 100, from 0.05 to 100, from 0.1 to 100, from 0.5 to 100, from 1 to 100, from 5 to 100, from 10 to 100, from 0.001 to 50, from 0.005 to 50, from 0.01 to 50, from 0.05 to 50, from 0.1 to 50, from 0.5 to 50, from 1 to 50, from 5 to 50, from 10 to 50, 0.001 to 10, from 0.005 to 10, from 0.01 to 10, from 0.05 to 10, from 0.1 to 10, from 0.5 to 10, from 1 to 10, 0.001 to 5, from 0.005 to 5, from 0.01 to 5, from 0.05 to 5, from 0.1 to 5, from 0.5 to 5, from 1 to 5, 0.001 to 1, from 0.005 to 1, from 0.01 to 1, from 0.05 to 1, or from 0.1 to 1 mg/cm² of the skin surface treated.

The present disclosure provides methods involving administration of the compound of formula (I) or salt thereof, a compound of formula (I) or salt thereof for use as a medicament, use of a compound of formula (I) or salt thereof for the manufacture of a medicament for treating a skin condition, disease or disorder in a subject, and compositions comprising the compound of formula (I) or salt thereof. The above discussion, and the embodiments described therein (e.g. in relation to the nature of the compounds of formula (I), dosage regimes, applications, and compositions) has been made mainly in the context of discussing methods and compositions of the present disclosure, That discussion applies equally to all aspects of the present disclosure, including those aspects relating to the compound of formula (I) or salt thereof for use as a medicament, and use of the compound of formula (I) or salt thereof for the manufacture of a medicament for treating a condition, disease or disorder in a subject.

### EXAMPLES

The following Examples illustrate the invention.

### Example 1: Preparation of Urolithin A

Urolithin A (4) was prepared in two steps starting from 2-bromo-5-methoxybenzoic acid 1 and resorcinol 2. The pure compound was obtained as a pale yellow powder.

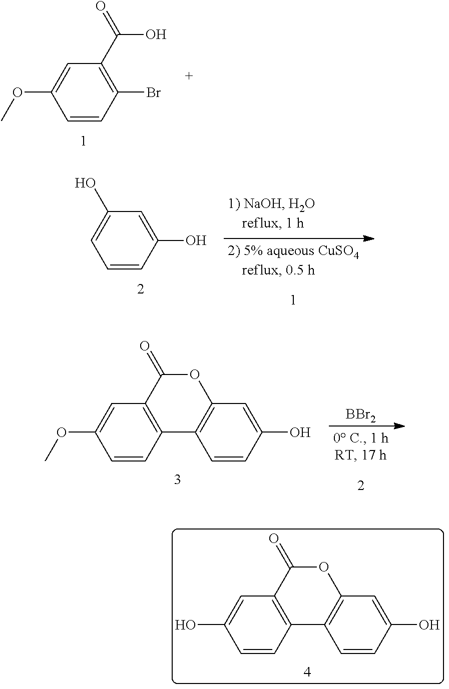

### Step 1:

A mixture of 2-bromo-5-methoxybenzoic acid 1 (27.6 g; 119 mmol; 1.0 eq.), resorcinol 2 (26.3 g; 239 mmol; 2.0 eq.) and sodium hydroxide (10.5 g; 263 mmol; 2.2 eq.) in water (120 mL) was heated under reflux for 1 hour. A 5% aqueous solution of copper sulphate (3.88 g of CuSO₄.5H₂O in 50 mL water; 15.5 mmol; 0.1 eq.) was then added and the mixture was refluxed for additional 30 minutes. The mixture was allowed to cool to room temperature and the solid was filtered on a Büchner filter. The residue was washed with cold water to give a pale red solid which was triturated in hot MeOH. The suspension was left overnight at 4° C. The resultant precipitate was filtered and washed with cold MeOH to yield the title compound 3 as a pale brown solid.

### Step 2:

To a suspension of 3 (10.0 g; 41 mmol; 1.0 eq.) in dry dichloromethane (100 mL) was added dropwise at 0° C. a 1 M solution of boron tribromide in dry dichloromethane (11.93 mL of pure BBr₃ in 110 mL of anhydrous dichloromethane; 124 mmol; 3.0 eq.). The mixture was left at 0° C. for 1 hour and was then allowed to warm up to room temperature. The solution was stirred at that temperature for 17 hours. Then ice was added thoroughly to the mixture. The yellow precipitate was filtered and washed with cold water to give a yellow solid which was heated to reflux in acetic acid for 3 hours. The hot solution was filtered quickly and the precipitate was washed with acetic acid, then with diethyl ether to yield the title compound 4 as a yellow solid. ¹H and ¹³C NMR were in accordance with the structure of 4.

### Example 2: Micronisation of Urolithin A

Urolithin A was micronized using an MC50 Spiral 20 Jetmill (Valortecs SAS, Blodelsheim, France), using filtered nitrogen, with a feed rate of 240 g/hr, a Venturi pressure of 12 bar, and a mill pressure of 12 bar. Urolithin A was micronized to give urolithin A having a particle size distribution of D₉₀=9 μm to 15 μm and D₅₀=2 to 9 μm. The actual particle size distribution was D₉₀=11.5 μm, D₅₀=3.9 μm, D₁₀=0.7 μm, measured using a Malvern Mastersizer 2000.

### Example 3: Urolithin A Skin Cream Composition

150 mg of micronized urolithin A was mixed with a spoonful (approximately 5 ml) of commercially available skin cream. The skin cream used was the one sold under the name Avene XeraCalm AD™, available from Pierre Fabre S.A. According to the manufacturer, the cream contains Avene thermal spring water (avene aqua), glycerin, mineral oil (paraffin in liquidum), cetearyl alcohol, Oenothera biennis (evening primrose) oil (Oenothera biennis oil), caprylic/capric triglyceride, cetearyl glucoside, aquaphilus dolomiae extract, arginine, carbomer, evening primrose oil/palm oil aminopropanediol esters, glycine, sodium hydroxide, tocopherol and water (aqua).

After mixing, a homogeneous mixture was obtained. The composition had a slightly darker colour than the skin cream starting product. The composition was smooth and of regular colour. The presence of the urolithin had no discernible effect on the texture of the skin cream product.

### Example 4: In Vitro Testing of Urolithin A on Skin Viability in EpiDerm™ Cultures

Pre-screen treatment conditions: A good skin lightener should inhibit melanin synthesis but not cause cytotoxicity to the tissue. The impact of Urolithin A on skin cells viability was performed with the EpiDerm tissue (EPI-200). The reconstructed human epidermal model EpiDerm™ (EPI-200, MatTek, Ashland, USA) consists of normal human-derived epidermal keratinocytes, which have been cultured to form a multilayered highly differentiated model of the human epidermis.

Tissues were grown in maintenance medium (EPI-100-LLMM available from the MatTek Corporation). Treatment was done basolaterally, i.e. with the test compounds dissolved in the maintenance medium, for a total duration of 96 hours. Two EpiDerm™ tissues (n=2) were used per condition, which were 1) water, 2) DMSO 0.5%, 3) Urolithin A 25 μM, 4) Urolithin A 50 μM and 5) Urolithin A 100 μM. Maintenance medium added or not in Urolithin A or DMSO was refreshed at 48 hours.

Cell viability is measured by dehydrogenase conversion of MTT [(3-4,5-dimethylthiazole-2-yl)2,5-diphenyltetrazoliumbromide], present in cell mitochondria, into a blue formazan salt that is quantitatively measured after extraction from tissues. The MTT assay was performed by transferring the tissues to 24-well plates containing MTT medium (1 mg/ml). After a 3 hr MTT incubation, the blue formazan salt formed by cellular mitochondria was extracted with 2.0 ml/tissue of isopropanol (extractant solution, part # MTT-100-EXT) and the optical density of the extracted formazan was determined using a spectrophotometer at 570 nm. Relative cell viability was calculated for each tissue as % of the mean of the negative control tissues.

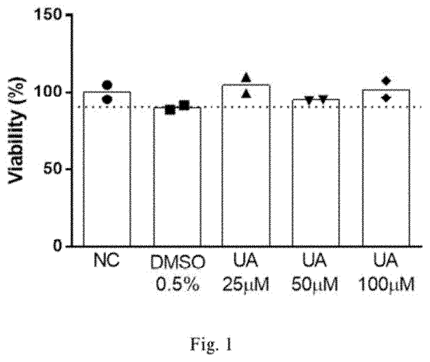

FIG. 1 shows the results of the viability assay performed in the EpiDerm™ tissues. Viability was higher than 90% for all the conditions, indicating that there is no cytotoxicity. This means that Urolithin A does not impair skin cell viability at the tested concentrations.

### Example 5: In Vitro Testing of Urolithin A on Melanogenesis in MelanoDerm™ Cultures

The MelanoDerm™ cultures from MatTek Corporation (Ashland, Mass., USA) are a pigmented 3D-Living Skin Equivalent model. MEL-300 tissues are obtained by seeding keratinocytes with melanocytes. MEL-300-B cultures contain melanocytes derived from a Black donor tissue and are used for the evaluation of skin lightening potential. MEL-300-B were prepared in a long life maintenance medium (EPI-100-LLMM available from the MatTek Corporation) and cultured for a total of 14 days. A total of four tissue replicates (n=4) was used per group.

Untreated tissue was used as negative control. Maintenance medium was refreshed every 2 days.

Urolithin A was applied to the samples by addition to the maintenance medium at a final concentration of 50 and 100 μM, prepared from a stock solution at 50 mM in DMSO. A vehicle control was prepared using DMSO at 0.2% in maintenance medium. Maintenance medium added in Urolithin A or DMSO was refreshed every 2 days. Kojic acid was used as a positive control at 2% in 50:50 butylene glycol:water and applied topically (i.e. on the stratum corneum, the outermost layer of the epidermis, of the MelanoDerm™ sample). Kojic acid is a well-known inhibitor of tyrosinase, a key enzyme that is responsible for melanogenesis in melanoma and melanocytes, and is used to treat hyperpigmentation, melasma, and wrinkle in cosmetics products. Every 2 days, tissues were rinsed with sterile PBS to remove Kojic acid prior to adding a fresh preparation of kojic acid at 2% in 50:50 butylene glycol:water. Maintenance medium was changed at the same time.

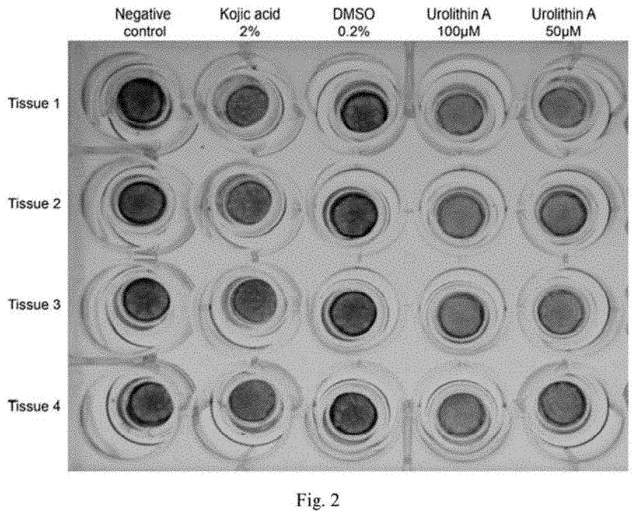

a. At day 14, all tissues were photographed by top view for macroscopic analysis of pigmentation using a Nikon Eclipse Ti microscope. FIG. 2 shows the obtained picture. From a macroscopic view, it is clear that tissues treated with Urolithin A at 50 and 100 μM are consistently lighter than the negative control and the DMSO 0.2% group. It is also visible that tissues treated with Urolithin A at 50 and 100 μM are as light as the tissues treated with the positive control Kojic acid applied topically at 2%.

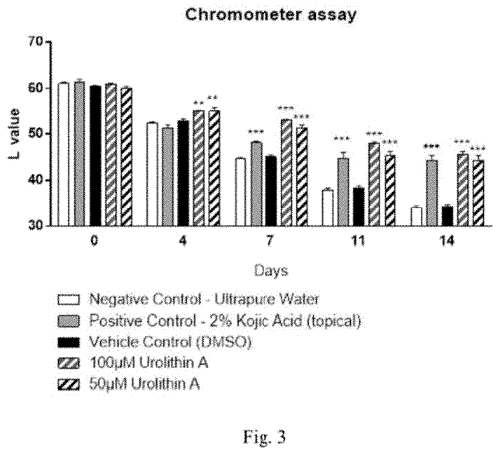

b. The lightness of the samples was assessed by measuring the L value using the Konica Minolta Color Spectrophotometer (CM-700d) at day 0, 4, 7, 11 and 14. The L value tells how light or dark the color is, with black corresponding to L=0 and white to L=100. FIG. 3 represents the L values measured in the difference samples. L value is significantly higher in the tissues treated with Urolithin A at 50 and 100 μM than the negative control group, starting from day 4 until day 14. It was not expected that Urolithin A 50 or 100 μM would have faster tissue-lightening effects than Kojic acid 0.2%. This is particularly visible at day 4 and 7, where Urolithin A treated tissues have a higher L value than the tissues treated with Kojic acid. At the end of the treatment, the L-values of tissues treated with Urolithin A and Kojic acid are equivalent, confirming what is visible in FIG. 2. Significant effects were observed at 50 μM and 100 μM, extrapolation from these results shows that in another embodiment of the invention lower concentrations of Urolithin A, within the range of 1 μM to 50 μM can be used in methods and composition of the invention.

c. Three tissues per group were used for the quantification of melanin. Tissues were first incubated in PBS to remove any phenol red remaining from the maintenance medium. Tissues were placed in a 1.7 ml microfuge tube with 500 μl of Solvable™ (Tissue and Gel Solubilizer 0.5 M—Packard BioScience Co. Catalogue No. 6NE9100) and incubated at 95° C. overnight along with melanin standards. Melanin standards were prepared by dissolving melanin (Sigma catalog number M8631) in Solvable™ at 1 mg/ml to make stock solution. Dilutions for the standard curve using the stock solution are given in Table 1.

TABLE 2
Dilutions of stock solution to make solutions for the standard curve

| Stock Solution | Solvable | Melanin Content |
|---|---|---|
| (μl) | (μl) | (μg) |
| — | 500 | 0 |
| 2.5 | 497.5 | 2.5 |
| 5 | 495 | 5 |
| 10 | 490 | 10 |
| 25 | 475 | 25 |
| 50 | 450 | 50 |
| 100 | 400 | 100 |

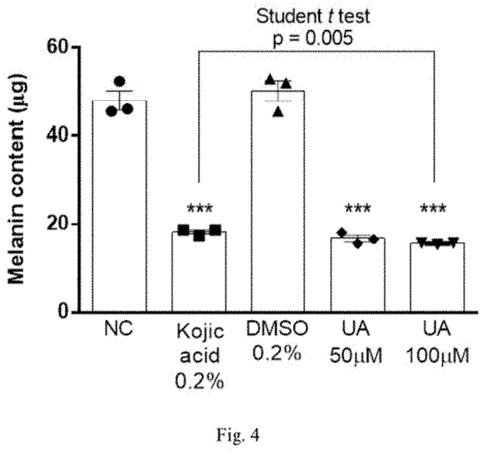

Following overnight incubation, samples were centrifuged at 13000 rpm for 5 minutes to pellet any insoluble material. 200 μl of each sample were transferred to a 96-well plate and read at 490 nm. FIG. 4 represents the results for the melanin content expressed in μg. Statistical significance was performed using a one-way ANOVA, followed by a Dunnett post-hoc test for multiple comparison of each group against the negative control. Both Urolithin A at 50 and 100 μM decrease significantly melanin content by 65 and 67% respectively, compared to the negative control. Kojic acid at 2% also decreases significantly the melanin content by 62%. In addition, the melanin content after treatment with Urolithin A at 100 μM is significantly lower than with Kojic acid at 2%, according to an additional Student t test to compare these two groups. These results are in agreement with the visual appreciation of lightness and the L values presented in FIGS. 2 and 3 respectively.

1. A method of preventing or treating a skin condition, disease, or disorder, comprising topically administering to skin of a subject in need thereof an effective amount of a compound of formula (I) 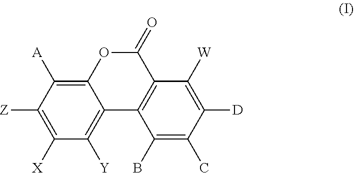
wherein:
A, B, C, D, W, X, Y and Z are each independently selected from H and OH;
or a salt thereof.

2. The method of claim 1, wherein the skin condition, disease, or disorder is selected from the group consisting of melasma, chloasma, mask of pregnancy, hyperpigmentation, skin-aging, liver spots, lentigo, inflammation of the skin, skin irritation, skin infection, warts, psoriasis, protection of skin from damage caused by the environment or therapy, melanosis, dermatitis, linea nigra, Addison's and Cushing's syndrome.

3. The method of in claim 2, wherein the skin condition, disease, or disorder is melasma; and the melasma is selected from the group consisting of stress-related melasma, pregnancy-related melasma, hypothyroidism-associated melasma, melasma associated with administration of an active ingredient, melasma associated with exposure to sunlight or UV light, and melasma associated with exposure to a chemical agent.

4. The method of claim 2, wherein the skin is protected from damage caused by sunlight or radiation therapy.

5. A method of skin bleaching, lightening skin colour, or lightening skin tone, comprising:
topically administering to skin of a subject in need thereof an effective amount of a compound of formula (I) 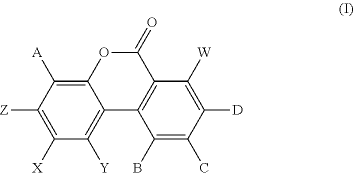
wherein:
A, B, C, D, W, X, Y and Z are each independently selected from H and OH;
or a salt thereof.

6. The method of claim 5, wherein the skin bleaching, skin colour lightening or skin tone lightening is cosmetic.

7. The method of claim 5, wherein the subject is healthy.

8. The method of claim 1, wherein the compound of formula (I) is urolithin A.

9. The method of claim 1, wherein the compound of formula (I) or salt thereof is administered in the form of a composition which comprises the compound of formula (I) or salt thereof and at least one excipient which is suitable for topical administration.

10. The method of claim 9, wherein the composition comprises 0.0001% to 1% of the compound of formula (I).

11. The method of claim 9 wherein the composition comprises one or more of the following:
(a) the composition is substantially free from polyphenols other than the compound of formula (I) or salt thereof;
(b) a micronized compound of formula (I);
(c) an organic solvent;
(d) a skin penetration enhancer;
(e) a lipid;
(f) mono-, di- and/or tri-glyceride;
(g) a medium chain triglyceride;
(h) less than 5% w/w water;
(i) at least one excipient which is unsuitable for oral administration;
(j) the composition is a semi-solid;
(k) the composition is a cream, paste or ointment;
(l) the composition is a skin cream;
(m) the composition is a sunscreen composition;
(n) a substance which is a physical and/or chemical sunscreen; and
(o) the compound of formula (I) or salt thereof is present in the composition in an amount in the range of from 0.005 to 20% w/w.

12. (canceled)

13. The method of claim 11, wherein the composition comprises a micronized compound of formula (I); and the micronized compound of formula (I) has a particle size distribution having a D₅₀ of 0.5 μm to 50 μm and a D₉₀ of 5 μm to 100 μm.

14. The method of claim 13, wherein the micronized compound of formula (I) has a particle size distribution having a D₅₀ less than 20 μm and a D₉₀ less than 30 μm.

15-24. (canceled)

25. A process for preparing a composition for topical administration, the composition comprising:
a) a compound of formula (I) 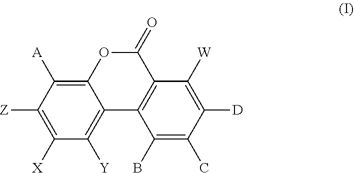
wherein:
A, B, C, D, W, X, Y and Z are each independently selected from H and OH;
or a salt thereof;
and
b) at least one excipient which is suitable for topical administration;
and the process comprising:
micronizing a compound of formula (I) or a salt thereof, and
admixing the micronized compound of formula (I) or salt thereof and at least one excipient suitable for topical administration.

26-28. (canceled)

29. The method of claim 5, wherein the compound of formula (I) is urolithin A.

30. The process of claim 25, wherein the compound of formula (I) is urolithin A.
